(19) United States
(12) Patent Application Publication
(10) Pub. No.: US 2004/0126354 A1
(43) Pub. Date: Jul. 1, 2004

(54) Nano carbon ball for deodorization

(76) Inventors: Jun-Yeob Song, Gyeonggi-do (KR); Jong-Yun Kim, Daejeon (KR); Seung-Kyu Park, Daejeon (KR); Jong-Sung Yu, Daejeon (KR); Yong-Ki Park, Daejeon (KR); Chul-Wee Lee, Daejeon (KR); Yun-Seog Kang, Daejeon (KR)

Correspondence Address: JONES DAY, 222 EAST 41ST ST, NEW YORK, NY, 10017, US

(21) Appl. No.: 10/684,352

(22) Filed: Oct. 10, 2003

(30) Foreign Application Priority Data
Dec. 27, 2002 (KR) 10-2002-0084983
Dec. 28, 2002 (KR) 10-2002-0085851
Dec. 28, 2002 (KR) 10-2002-0085852

(51) Int. Cl. A61L 9/00; A61L 9/01

(52) U.S. Cl. 424/76.1

(57) ABSTRACT

Disclosed is a nano carbon ball for deodorization composed of porous carbon shells having a spherical hollow core. At least one metal selected from the group consisting of transition metal, oxidized transition metal and alkali metal salt is impregnated to the shell. This nano carbon ball for deodorization may adsorb various kinds of malodor substances together with good deodorizing capability. Thus, the nano carbon ball may give excellent deodorizing effects by capturing and resolving the malodor substances when it used as a deodorant for various daily necessaries or in houses, offices, industrial facilities and other various stinking circumstances.

### BACKGROUND OF THE INVENTION

1. Field of the Invention

The present invention relates to a nano carbon ball for deodorization, and more particularly to a metal-impregnated nano carbon ball which is composed of a porous carbon shell having a spherical hollow core and used for deodorization.

2. Description of the Related Art

Generally, various bad smells are generated from daily necessaries such as refrigerator, air conditioner, diaper, hygienic band, cigarette, footwear cabinet and clothes chest and in daily life area such as bedroom, bathroom and automobile room. In addition, bad smells are also generated from exhaust gas of automobiles and industrial equipments such as refuse disposal plant, wastewater disposal plant and factories. Materials generating bad smells are representatively as follows: methanthiol, methyl sulfide, dimethyl disulfide, hydrogensulfide, ammonia, trimethylamine, acetaldehyde, nitric oxide, nitrous oxide, styrene and so on.

Also, various kinds of deodorizing agents have been developed in order to eliminate such bad smells.

Recently, a method for making a nano carbon ball composed of a porous carbon shell having a spherical hollow core (Adv. Mater. 2002, 14, no. 1, January 4) is proposed. This nano carbon ball has the advantage that it may adsorb more various kinds of malodor substances than a conventional activated carbon deodorizing agent. However, the nano carbon ball has some limitations that it may not adsorb any more malodor substances after adsorbing a certain amount. In addition, the above-mentioned nano carbon ball is a limited capacity in deodorizing.

### SUMMARY OF THE INVENTION

The present invention is designed to solve such drawbacks of the prior art, and therefore an object of the present invention is to provide a nano carbon ball having excellent deodorizing ability and capable of adsorbing various kinds of malodor substances.

In order to accomplish the above object, the present invention provides a nano carbon ball for deodorization comprising a porous carbon shell having a spherical hollow core, wherein at least one metal selected from the group consisting of transition metal, oxidized transition metal and alkali metal salt is impregnated to the shell.

Preferably, the transition metal may be selected from the group consisting of Copper (Cu), Iron (Fe), Manganese (Mn), Nickel (Ni), Cobalt (Co), Silver (Ag), Gold (Au), Vanadium (V), Ruthenium (Re), Titanium (Ti), Chrome (Cr), Zinc (Zn) and Palladium (Pd), and the alkali metal salt may be selected from the group consisting of sodium bromide (NaBr), sodium iodide (Nal), potassium bromide (KBr), potassium iodide (KI) and potassium iodate (KIO₃).

### BRIEF DESCRIPTION OF THE DRAWINGS

Other objects and aspects of the present invention will become apparent from the following description of embodiments with reference to the accompanying drawing in which:

FIG. 1 is a schematic diagram for illustrating the process for making a nano carbon ball for deodorization according to the present invention; and

FIGS. 2 and 3 are graphs showing deodorizing effects of the nano carbon ball for deodorization according to the present invention to ammonia and methanthiol.

### DESCRIPTION OF THE PREFERRED EMBODIMENT

Hereinafter, embodiments of the present invention will be described, however the present invention is not limited to the following embodiments but capable of being modified in diverse ways within the scope of the invention.

A nano carbon ball of the present invention has a ball-shaped carbon structure composed of a hollow core and a porous shell. To the shell, transition metal, oxidized transition metal, alkali metal salt or their mixture is impregnated. The mesoporous pores formed in the shell may not only adsorb various kinds of malodor substances but also chemically adsorb and destroy the malodor substances for deodorization. This nano carbon ball for deodorization of the present invention shows more excellent deodorizing ability than the impregnated activated carbon disclosed in Korean Laid-open Patent Publication No. 1999-80808. In other words, since the activated carbon has microporous pores, the pores may be clogged and deteriorate the deodorizing ability when the deodorizing materials are impregnated. However, since the nano carbon ball for deodorization of the present invention has the mesoporous pores in the shell, such a problem is not caused. In addition, since the malodor substances are captured in the hollow core of the nano carbon ball for deodorization of the present invention, differently to the impregnated activated carbon, it is possible to give sufficient contact time between the malodor substances and the deodorizing material impregnated on the inner surface of the shell. In addition, the nano carbon ball of the present invention may prevent secondary pollution caused when decomposition products generated by the deodorizing materials are emitted outside.

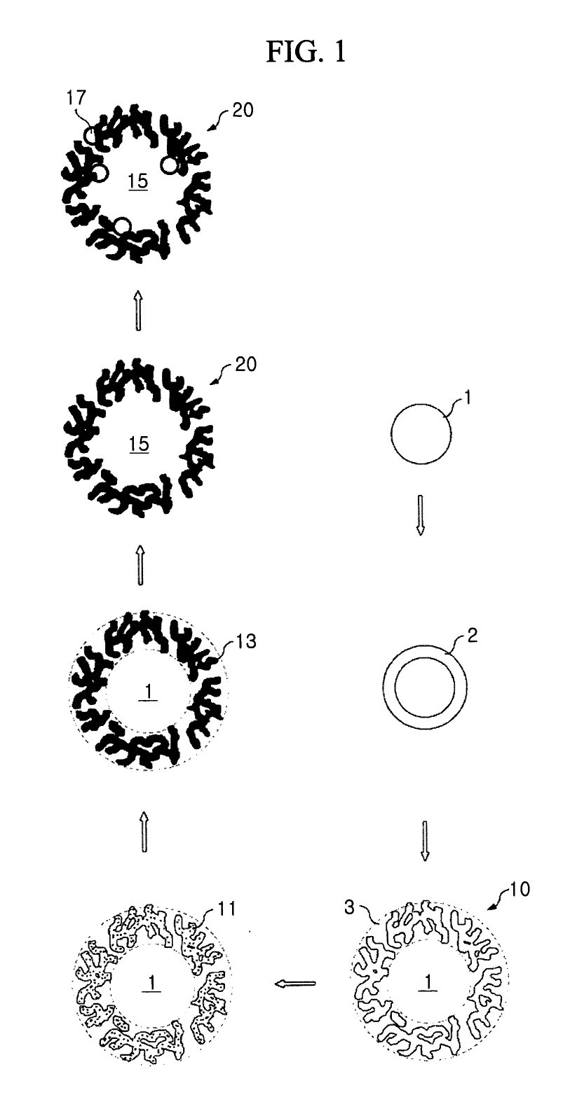

An example of a method for making the nano carbon ball for deodorization according to the present invention is described in detail with reference to FIG. 1.

At first, a spherical silica core is prepared. The silica core 1 may be composed according to the well-known Stober method (Stober, W., Fink, A.; Bohn, E. J. Colloid Inter. Sci. 1968, 26, 62) from a silica precursor such as tetramethylorthosilicate and tetraethylorthosilicate. The silica core preferably has a diameter of 10˜1,000 nm.

After that, a shell 2 is grown up on the surface of the silica core 1 by using silica precursor and surface active agent such as alkyltrimethoxysilane expressed as the following Chemical Formula 1, alkyltriethoxysilane expressed as the following Chemical Formula 2, halogenated alkyltrimethylammonium expressed as the following Chemical Formula 3, alkylpolyoxyethylene expressed as the following Chemical Formula 4 and glycerolethoxylate expressed as the following Chemical Formula 5, in a solvent.

Chemical Formula 1

R₁R₂R₃R₄Si(OCH₃)₃

In the Chemical Formula 1, R₁, R₂ and R₃ are methyl groups, and R₄ is an alkyl group having a carbon number of 12 to 22.

Chemical Formula 2

R₁R₂R₃R₄Si(OC₂H₅)₃

In the Chemical Formula 2, R₁, R₂ and R₃ are ethyl groups, and R₄ is an alkyl group having a carbon number of 12 to 22.

Chemical Formula 3

R₁R₂R₃R₄NX

In the Chemical Formula 3, R₁, R₂ and R₃ are independently methyl or ethyl groups, R₄ is an alkyl group having a carbon number of 12 to 22, and X is halogen.

Chemical Formula 4

R(OCH_{2 CH}₂)nOH

In the Chemical Formula 4, R is an alkyl group having a carbon number of 12 to 22, and n is an integer in the range of 3˜20.

Chemical Formula 5

CH₂(CH₂O)n₁HCH(CH₂O)n₂HCH₂(CH₂O)n₃H

In the Chemical Formula 5, n₁, n₂ and n₃ are independently integers in the range of 4˜20.

And then, after the products having the shell is selectively filtered and calcined at, for example, 500˜600° C. to remove the surface active agent components. Then, particles 10 having a silica shell 3 are obtained in which mesoporous pores having a certain size are formed in the place where the surface active agent is removed. The size of the mesoporous pore and the thickness of the shell may be controlled by changing the kind of the surface active agent and the kind and molecular ratio of the silica precursor. The porous carbon shell preferably has a thickness of 50˜500 nm.

Subsequently, a monomer 11, such as acrylonitrile, phenol-formaldehyde and divinylbenzene, which is capable of forming polymer, is injected into the mesoporous pores formed in the shell. After that, the monomer is polymerized to form a carbon precursor and the particle 10 having the silica shell is formed. Preferably, the monomer is polymerized using the condensation polymerization or radical polymerization. In addition, in the radical polymerization, the monomer is sufficiently mixed with the radical initiator and the injected into the mesoporous pores of the silica particle, and then polymerized according to the characteristics of the monomer. At this time, as for a radical initiator, for example, azobisisobutyronitrile (AIBN), t-butyl peracetate, benzoyl peroxide, acetyl peroxide and lauryl peroxide may be used. This polymerization is well known in the art, and preferably conducted for about 12 hours at 60˜130° C. to make the silica structure containing polymer.

And then, the treatment of the silica structure containing the polymer (or, carbon precursor) under the nitrogen atmosphere at about 1,000° C. makes the silica structure contain a carbonized polymer 13. After that, a nano carbon ball 20 having a ball shape which has a spherical hollow core 15 and a porous carbon shell is obtained as a consequence of etching the carbonized silica structure by hydrofluoric acid solution or sodium hydroxide/ethyl alcohol mixed solution.

After that, the nano carbon ball 20 is dipped into an aqueous solution composed of transition metal, oxidized transition metal, alkali metal salt or their mixture and matured at the room temperature for 2˜3 days, and then filtered and dried at 70˜110° C. to make the metal-impregnated nano carbon ball according to the present invention. As for the transition metal or the oxidized transition metal which may be impregnated to the shell, Copper (Cu), Iron (Fe), Manganese (Mn), Nickel (Ni), Cobalt (Co), Silver (Ag), Gold (Au), Vanadium (V), Ruthenium (Re), Titanium (Ti), Chrome (Cr), Zinc (Zn), Palladium (Pd) or their oxide may be used. As for the alkali metal, sodium bromide (NaBr), sodium iodide (NaI), potassium bromide (KBr), potassium iodide (KI) and potassium iodate (KIO₃) may also be used. An impregnated amount of the metal may be controlled by changing the concentration of the metal-containing aqueous solution or the infiltration time, and is preferably 0.01˜30 wt % on the basis of the total weight of the nano carbon ball for deodorization.

The metal-impregnated nano carbon ball according to the present invention may be provided with one or more kinds of metals among the above-mentioned metals. Thus, the deodorizing agent containing the metal-impregnated nano carbon ball according to the present invention may be prepared or composed in various ways depending on the kind of bad smell or its usage. For example, a deodorizing agent may contain nano carbon ball to which only one kind of metal is impregnated, or to which two different kinds of metals are impregnated, or more than two kinds of metals are impregnated.

The metal-impregnated nano carbon ball of the present invention may be used for deodorizing and eliminating various odor materials such as methanthiol, methyl sulfide, dimethyl disulfide, hydrogen sulfide, ammonia, trimethylamine, styrene, acetaldehyde, nitric oxide, nitrous oxide, indoor bad smells generated in bathroom, kitchen or footwear cabinet in home, and smell of tobacco. Thus it also may give excellent effects in eliminating bad smells of refrigerator, air conditioner, air cleaner, automobile room, exhaust gas of cars as well as a human body.

In addition, the metal-impregnated nano carbon ball of the present invention may be uniformly dispersed and stuck to one having a shape of sheet, pack or pad, thus it may be applied to goods such as a diaper for the infant or the person suffered from the incontinence or a hygienic band for women which use such matters.

Embodiment 1 to 4

A spherical silica core is composed according to the well-known Stober method by using tetraethoxysilane as a silica precursor. At this time, tetraethoxysilane is put therein and reacted together with octadecyltrimethoxysilane (C₁₈-TMS), and then filtered to obtain silica particles. The silica particles are thermally treated at 550° C. for 5 hours so that mesoporous pores having a certain size are formed in the place where the surface active agent is removed. Then, divinylbenzene is sufficiently mixed with azobisisobutyronitrile (AIBN), which is a radical initiator, and the injected into the mesoporous pores of the silica particle, and then polymerized at 130° C. for about 12 hours to make a silica structure containing polymer. In succession, the silica structure containing polymer is carbonized under nitrogen circumstance at 1,000° C. to form a carbon/silica composite. Subsequently, the carbon/silica composite is put into hydrofluoric acid to remove inorganic structure of the carbon/silica composite, therefore the nano carbon ball which has a ball-shaped carbon structure including a hollow core and a porous shell is obtained.

After that, in order to impregnate the metals of the following Table 1, the nano carbon ball made along with the above-mentioned method is dipped into the IN of metal solution for about 50 hours, filtered and dried at 70° C., therefore a metal-impregnated nano carbon ball is obtained.

Among the metal-impregnated nano carbon ball A˜H, 0.01 g of A, D, E and H are picked as Embodiments 1˜4, respectively.

TABLE 1

| | The kind of impregnated metal |
|---|---|
| | (impregnated amount of metal, %) |
| A | copper (1.3) + manganese (0.3) |
| B | nickel (3.1) + iron (0.8) |
| C | gold (0.8) + chrome (0.9) + palladium (0.8) |
| D | copper (3.1) + iron (0.8) + zinc (0.8) |
| E | potassium iodide (3.4) |
| F | silver (4.2) |
| G | cobalt (2.1) + potassium iodate (1.3) |
| H | vanadium (2.1) + ruthenium (0.3) + titanium (0.6) |

In Table 1, Impregnated Amount (%) of Metal = Weight of Metal/Weight of Nano carbon ball × 100.

### COMPARATIVE EXAMPLE 1

A nano carbon ball to which no metal is impregnated is made in the same way of the above embodiments, and 0.01 g is taken from the nano carbon ball as Comparative Example 1.

### COMPARATIVE EXAMPLE 2

A nano carbon ball to which no metal is impregnated is made in the same way of the above embodiments, and 0.1 g is taken from the nano carbon ball as Comparative Example 2.

### COMPARATIVE EXAMPLE 3

0.1 g of activated carbon (manufactured by Junsei in Japan) is taken as Comparative Example 3.

### COMPARATIVE EXAMPLES 4˜8

0.1 g of various commercially-used conventional deodorizers are taken as Comparative Examples 4˜8.

### COMPARATIVE EXAMPLES 9˜12

4 kinds of metal-impregnated activated carbons made by impregnating metals to the activated carbon (manufactured by Junsei in Japan) with same metal compositions as Embodiments 1 to 4 are respectively taken as Comparative Examples 9˜12.

In the estimation of the deodorizing effects, trimethylamine, ammonia, methanthiol and acetaldehyde are used as an odor source. At first, deodorizing agents having a weight shown in Table 2 are respectively put into a 250 ml transparent container which contains malodor substances such as 0.2 ml of ammonia 0.1% solution, 0.07 ml of trimethylamine 1% solution, 0.15 ml of acetaldehyde 1% solution and 0.12 ml of methanthiol 0.1% benzene solution. After that, the container is sealed with a cap in which a detecting tube for measuring a residual amount of the malodor substances is attached, and left alone for 30 minutes. After that, with passing the gas in the container through the detecting tube, the color change of the detecting tube is monitored to measure the capability of the deodorizing agent.

This capability (%) of the deodorizing agent is calculated according to the following formula on the basis of the blank test in which only the odor source is put without the deodorizing agent, and the calculated results are shown in Table 2.

TABLE 2
Deodorizing Capability (%) = [(Detecting Tube Value of Blank Test
(ppm) − Measured Detecting Tube Value (ppm))/
(Detecting Tube Value of Blank Test (ppm))] × 100

| | | Deodorizing Capability (%) | | | |
|---|---|---|---|---|---|
| | | | Tri- | | |
| | Deodorizing agent | Ammo- | methyl- | Acetal- | Methan- |
| | (weight) | nia | amine | dehyde | thiol |
| Embodiment | Metal-impregnated | 78 | 87 | 63 | 90 |
| 1 | nano carbon ball | | | | |
| | (0.01 g) | | | | |
| Embodiment | Metal-impregnated | 98 | 94 | 47 | 93 |
| 2 | nano carbon ball | | | | |
| | (0.01 g) | | | | |
| Embodiment | Metal-impregnated | 89 | 92 | 54 | 98 |
| 3 | nano carbon ball | | | | |
| | (0.01 g) | | | | |
| Embodiment | Metal-impregnated | 88 | 86 | 58 | 92 |
| 4 | nano carbon ball | | | | |
| | (0.01 g) | | | | |
| Comparative | Nano carbon ball | 43 | 85 | 7 | 29 |
| example 1 | (0.01 g) | | | | |
| Comparative | Nano carbon ball | 80 | 84 | 40 | 91 |
| example 2 | (0.1 g) | | | | |
| Comparative | Activated carbon | 61 | 58 | 20 | 48 |
| example 3 | (0.1 g) | | | | |
| Comparative | β-Cyclodextrin | 15 | 2 | 0 | 0 |
| example 4 | (0.1 g) | | | | |
| Comparative | Copper chloride | 68 | 85 | 4 | 84 |
| example 5 | (98%, 0.1 g) | | | | |
| Comparative | Carbazole W7 | 60 | 9 | 12 | 0 |
| example 6 | MCT (0.1 g) | | | | |
| Comparative | Aluminum chloride | 92 | 97 | 0 | 0 |
| example 7 | (0.1 g) | | | | |
| Comparative | Zeolites | 30 | 27 | 8 | 0 |
| example 8 | (0.1 g) | | | | |
| Comparative | Metal-impregnated | 76 | 67 | 34 | 68 |
| example 9 | activated carbon | | | | |
| | (0.01 g) | | | | |
| Comparative | Metal-impregnated | 78 | 58 | 40 | 64 |
| example 10 | activated carbon | | | | |
| | (0.01 g) | | | | |
| Comparative | Metal-impregnated | 69 | 66 | 47 | 72 |
| example 11 | activated carbon | | | | |
| | (0.01 g) | | | | |
| Comparative | Metal-impregnated | 70 | 69 | 45 | 70 |
| example 12 | activated carbon | | | | |
| | (0.01 g) | | | | |

From Table 2, it will be understood that the nano carbon ball for deodorization according to the embodiments of the present invention shows excellent deodorizing effects against 4 kinds of odorizing sources, compared with the comparative examples, even though a small amount as much as 10% (0.01 g) is used. In addition, it will be also understood from Table 2 that the deodorizing effects of the metal-impregnated nano carbon ball increase about 20% or above for the four odorizing sources.

Embodiment 5

4 types of metal-impregnated nano carbon balls are made according to the same way as the Embodiment 1 with the metals shown in the following Table 3, respectively. Then, deodorizing filter for refrigerator is prepared by mixing the 4 kinds of metal-impregnated nano carbon ball with a polypropylene polymer binder as much as 20 wt % of the total filter weight, respectively and executing further treatment. Among the metal-impregnated nano carbon balls I˜K, K is taken as Embodiment 5.

### COMPARATIVE EXAMPLE 13

The Comparative Example 13 is identical to Embodiment 5, except that an metal-impregnated activated carbon made by impregnating the metals to the activated carbon (manufactured by Junsei in Japan) is used instead of the nano carbon ball of Embodiment 5.

TABLE 3

| | Kind of impregnated metal |
|---|---|
| | (impregnated amount of metal, %) |
| I | copper (1.3) + manganese (0.3) |
| J | cobalt (2.1) + iron (0.8) + zinc (0.3) |
| K | potassium iodide (3.2) + copper (0.8) |

In Table 3, Impregnated Amount of Metal (%) = Weight of Metal/Weight of Nano carbon ball × 100

The test of deodorizing effects is conducted under the experimental condition as shown in the following Table 4.

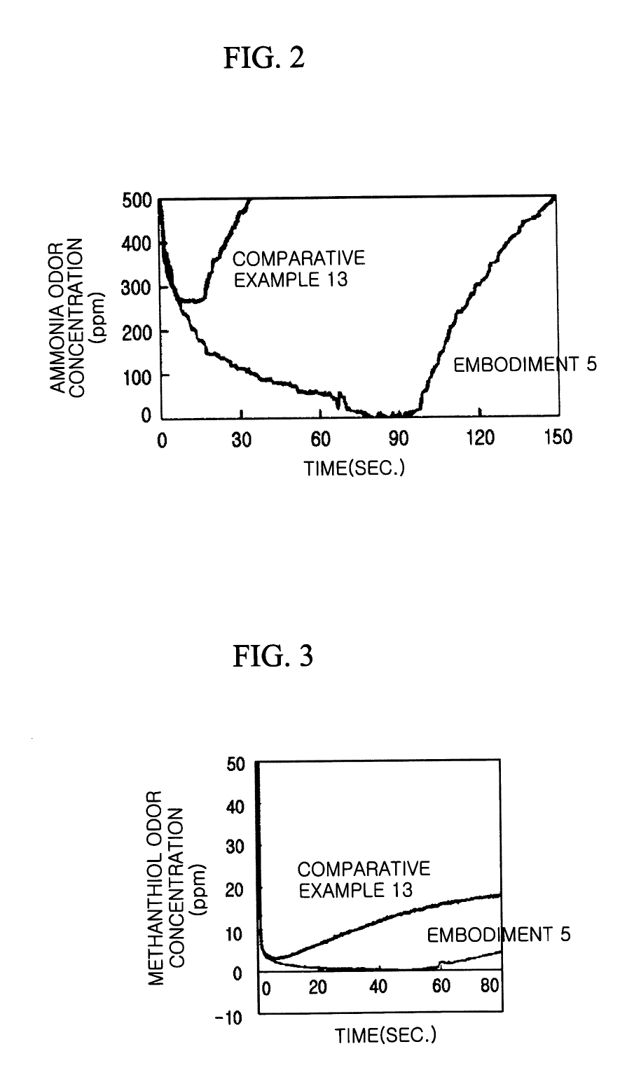

In case of the deodorizing effect test for ammonia, 0.5 g of the deodorizing filters of Embodiment 5 and Comparative Example 13 are put into tube reactors, respectively. After that, the ammonia gas is flowed through each tube reactor, and then the concentrations of the discharged gas are analyzed, of which results are expressed in Table 5 and FIG. 2.

In case of the deodorizing effect test for methanthiol, 0.01 g of the deodorizing agents of Embodiment 5 and Comparative Example 13 are put into tube reactors, respectively. After that, the methanthiol gas is flowed through each tube reactor, and then the concentrations of the discharged gas are analyzed, of which results are expressed in Table 5 and FIG. 3.

At this time, a gas chromatograph or a mass spectrometer is used as an analyzer, and the relative humidity is 50%.

TABLE 4

| | | | | | Initial |
|---|---|---|---|---|---|
| | | | | | gas |
| | Adsorption | Relative | Gas flow | Amount of | concen- |
| Odor | temperature | humidity | rate | adsorbent | tration |
| sources | (° C.) | (RH) | (cc/min) | (g) | (ppm) |
| Ammonia | Room | 50% | 600 | 0.5 | 500 |
| | temperature | | | | |
| Methanthiol | Room | 50% | 100 | 0.01 | 50 |
| | temperature | | | | |

Here, in order to analyze the odor gas concentration, the concentrations of the discharged gas of the comparative example and the embodiment are comparatively measured. The dynamic test was carried out to evaluate the capacity of carbon for removal of odor substances, based on ASTM D28 as a standard test method. Break-point is defined as breakthrough time in the breakthrough curve.

TABLE 5

| | | | Deodorizing effect | |
|---|---|---|---|---|
| | | | | Concen- |
| | | | | tration |
| | | | Break- | at break- |
| | Deodorizing agent | Odor | point | point |
| | (weight) | source | (min.) | (ppm) |
| Embodiment 5 | Metal-impregnated | Ammonia | 80˜90 | 0 |
| | nano carbon ball | | | |
| | (0.5 g) | | | |
| Comparative | Metal-impregnated | Ammonia | 8˜16 | 260 |
| example 13 | activated carbon | | | |
| | (0.5 g) | | | |
| Embodiment 5 | Metal-impreganted | Methanthiol | 50˜55 | 0 |
| | nano carbon ball | | | |
| | (0.01 g) | | | |
| Comparative | Metal-impregnated | Methanthiol | 3˜6 | 3 |
| example 13 | activated carbon | | | |
| | (0.01 g) | | | |

It will be known from Table 5, FIG. 2 and FIG. 3 that the deodorizing agent composition of Embodiment 5 takes a longer time to reach the break-point than the conventional deodorizing agent of Comparative Example 13. In case of ammonia at the break-point, ammonia gas is not detected in Embodiment 5, but is detected to have a concentration of 260 ppm in Comparative Example 13. And in case of methanthiol gas at the break-point, methanthiol gas is not detected in Embodiment 5, but is detected to have a concentration of 3 ppm in Comparative Example 13. Thus, it will be understood from the results that the deodorizing agent of the present invention has much more excellent deodorizing ability in eliminating odors like the ammonia gas and methanthiol than the conventional one.

### APPLICABILITY TO THE INDUSTRY

As described above, the metal-impregnated nano carbon ball according to the present invention adsorbs various kinds of malodor substances as well as shows good deodorizing ability. Thus, the nano carbon ball of the present invention may show excellent deodorizing effect in capturing and resolving the malodor substances when it is used as an deodorizing agent in various odorizing daily necessaries, living spaces, industrial spots and other various stink-generating circumstances.

The present invention has been described in detail. However, it should be understood that the detailed description and specific examples, while indicating preferred embodiments of the invention, are given by way of illustration only, since various changes and modifications within the spirit and scope of the invention will become apparent to those skilled in the art from this detailed description.

What is claimed is:

1. A nano carbon ball for deodorization comprising a porous carbon shell having a spherical hollow core, wherein at least one metal selected from the group consisting of transition metal, oxidized transition metal and alkali metal salt is impregnated to the shell.

2. A nano carbon ball for deodorization according to claim 1,
wherein the transition metal is one selected from the group consisting of Copper (Cu), Iron (Fe), Manganese (Mn), Nickel (Ni), Cobalt (Co), Silver (Ag), Gold (Au), Vanadium (V), Ruthenium (Re), Titanium (Ti), Chrome (Cr), Zinc (Zn) and Palladium (Pd), and
wherein the alkali metal salt is one selected from the group consisting of sodium bromide (NaBr), sodium iodide (NaI), potassium bromide (KBr), potassium iodide (KI) and potassium iodate (KIO₃).

3. A nano carbon ball for deodorization according to claim 1 or 2,
wherein an impregnated amount of the metal is 0.01˜30 wt % on the basis of the total weight of the nano carbon ball for deodorization.

4. A nano carbon ball for deodorization according to claim 1 or 2,
wherein the spherical hollow core has a diameter of 10˜1,000 nm, and the porous carbon shell has a thickness of 50˜500 nm.
